(19) United States
(12) Patent Application Publication
(10) Pub. No.: US 2009/0076395 A1
(43) Pub. Date: Mar. 19, 2009

(54) Optimized intravascular ultrasound probe catherers

(75) Inventor: E. Ignacio Cespedes, Folsom, NJ (US)

Correspondence Address: PATTON BOGGS LLP, 8484 WESTPARK DRIVE, SUITE 900, MCLEAN, VA, 22102, US

(73) Assignee: Prescient Medical, Inc., Doylestown, PA (US)

(21) Appl. No.: 11/902,170

(22) Filed: Sep. 19, 2007

(51) Int. Cl. A61B 8/12

(52) U.S. Cl. 600/462

(57) ABSTRACT

The present invention provides intravascular diagnostic catheters that include a centrally disposed intravascular ultrasound (IVUS) imaging element and radially extendable, wall-contacting optical probe arms of a novel design for improving the IVUS imaging aspect of combined IVUS and optical analysis. In one embodiment, the dimension of the portion of the probe arm(s) that is in the field-of-view of the IVUS imaging element is minimized. In another embodiment, thin tethers are disposed in the field-of-view of the IVUS imaging element, rather than a continuation of the probe arm itself into the IVUS field. In still another embodiment, the probe arms at least substantially do not impinge on the field-of-view of the IVUS imaging element and tethers that are not within the field-of-view of the IVUS imaging element are used to control the probe arms, thereby providing a clear field-of-view for the IVUS imaging element.

### FIELD OF THE INVENTION

The invention relates generally to the fields of catheter-based intravascular ultrasound (IVUS) and intravascular optical spectroscopy.

### BACKGROUND OF INVENTION

Various modalities for diagnostically interrogating blood vessel walls to locate and characterize atherosclerotic lesions have been previously proposed including intravascular ultrasound (IVUS) and optical spectroscopic techniques, such as Raman spectroscopy. IVUS catheters have generally fallen into two categories: a single transducer that is rotated about a central axis or an array of elements that are phased (controlled delays) relative to one another on excitation or collection to provide spatial information. Piezoelectric effect-based ultrasound detectors are well known in the art. More recently, optics-based ultrasound sensors, such as Fabry-Perot interferometers, have also been described.

The following patents and publications are also background to the present invention.

U.S. Pat. No. 5,840,023 discloses systems and methods of acoustic imaging for medical diagnosis, and is incorporated by reference herein in its entirety.

U.S. Pat. No. 6,281,976 discloses fiber-optic Fabry-Perot interferometer sensors and methods of measurement therewith, and is incorporated by reference herein in its entirety.

U.S. Pat. No. 6,445,939 discloses ultra-small optical probes that include an optical fiber and a lens that has at least substantially the same diameter as the fiber and which may be in communication with a beam director, and is incorporated by reference herein in its entirety.

U.S. Pat. No. 6,522,913 discloses systems and methods for visualizing tissue during diagnostic or therapeutic procedures that utilize a support structure that brings sensors into contact with the lumen wall of a blood vessel, and is incorporated by reference herein in its entirety.

U.S. Pat. No. 6,701,181 discloses multi-path optical catheters, and is incorporated by reference herein in its entirety.

U.S. Pat. No. 6,813,401 discloses methods for fabricating Fabry-Perot polymer film sensing interferometers on optical fiber substrates, and is incorporated by reference herein in its entirety.

U.S. Pat. No. 6,873,868 discloses multi-fiber catheter probe arrangements for tissue analysis or treatment, and is incorporated by reference herein in its entirety.

U.S. Pat. No. 6,839,496 discloses optical fiber probes for photoacoustic material analysis, and is incorporated by reference herein in its entirety.

U.S. Pat. No. 6,949,072 discloses devices for vulnerable plaque detection that combine IVUS and optical analysis, and is incorporated by reference herein in its entirety.

U.S. Publication No. 2002/0183622 discloses a fiber-optic apparatus and method for the optical imaging of tissue samples, and is incorporated by reference herein in its entirety.

U.S. Publication No 2003/0032880 discloses apparatuses and methods for ultrasonically identifying vulnerable plaques, and is incorporated by reference herein in its entirety.

U.S. Publication No. 2003/0125630 discloses catheter probe arrangements for tissue analysis by radiant energy delivery and radiant energy collection, and is incorporated by reference herein in its entirety.

Each of U.S. Publication Nos. 2003/0199747, 2003/0199767 and 2003/0199768 discloses a basket catheter having a centrally disposed intravascular ultrasound imaging element and peripheral optical thermography sensors on the basket arms, and is incorporated by reference herein in its entirety.

U.S. Publication No. 2004/0260182 discloses intraluminal spectroscope devices with wall-contacting probes, and is incorporated by reference herein in its entirety.

U.S. Publication No. 2005/0054934 discloses an optical catheter with dual-stage beam redirector, and is incorporated by reference herein in its entirety.

U.S. Publication No. 2005/0165315 discloses a side-firing fiber-optic array probe, and is incorporated by reference herein in its entirety.

U.S. Publication No. 2006/0139633 discloses the use of high wavenumber Raman spectroscopy for evaluating tissue, and is incorporated by reference herein in its entirety.

In view of the above, what is needed and desirable are new and improved apparatuses and methods for the intravascular evaluation of blood vessel walls using ultrasound in combination with optical analysis.

### SUMMARY OF INVENTION

The invention provides intravascular diagnostic catheters including a centrally disposed (e.g., on or around a central shaft of the catheter) intravascular ultrasound (IVUS) imaging element and radially extendable, wall-contacting/wall-approaching optical probe arms, where the probe arms are designed to improve the IVUS imaging obtained in the combined IVUS and optical analysis.

One embodiment of the invention provides a basket-type intravascular diagnostic catheter that includes:
a proximal end and a distal insertion end;
a basket section disposed at or near the distal insertion end of the catheter, said basket section including at least one, such as at least two, radially extendable wall-contacting/wall-approaching optical probe arms each having a proximal end and a distal end and a wall-contacting/wall-approaching portion disposed therebetween and each having at least one optical fiber entering an end of the probe arm and terminating in the wall-contacting/wall-approaching portion thereof, the optical fibers of each probe arm all entering from the distal ends of the probe arms or all entering from the proximal ends of the probe arms;
a radially centrally disposed, radially scanning IVUS imaging element disposed within the basket section and having a field-of-view,
the probe arms extending laterally over the IVUS imaging element, a first lateral portion of each probe arm being in the field-of-view of the IVUS imaging element and a second lateral portion of each probe arm being outside of the field-of-view of the IVUS imaging element,
the cross-sectional dimension of the first lateral portion of the probe arms being smaller than the cross-sectional dimension of the second lateral portion of the probe arms.

Another embodiment of the invention provides an intravascular diagnostic catheter employing tethered probe arms that includes:
a proximal end and a distal insertion end;
an interrogation section disposed at or near the distal insertion end of the catheter, said interrogation section including at least one, such as at least two, radially extendable wall-contacting/wall-approaching optical probe arms each having a wall-contacting/wall-approaching portion disposed at an end thereof and each having at least one optical fiber entering the probe arm and terminating at or near the wall-contacting/wall-approaching portion/end; and
a radially centrally disposed, radially scanning IVUS imaging element laterally adjacent to the wall-contacting/wall-approaching ends of the optical probe arms in their radially extended state,
the wall-contacting/wall-approaching end of each probe arm being connected by a flexible tether line to a radially central position on the catheter.

In one variation, the cross-sectional dimension of the flexible tether lines is smaller than the cross-sectional dimension of the optical probe arms.

In another variation of this embodiment each of the flexible tether lines laterally traverses the IVUS imaging element before connecting to a radially central position of the catheter and the cross-sectional dimension of the flexible tether lines is smaller than the cross-sectional dimension of the optical probe arms. The tether lines may be extendable and retractable to control the probe arms

In another variation of this embodiment, each of the flexible tether lines connects the wall-contacting/wall-approaching end of an optical probe arm to a radially central position on the catheter at lateral position within the lateral dimension of the probe arm in its radially extended state, i.e., not extending beyond the lateral position of the wall-contacting/wall-approaching ends of the probe arms. Thus, the tethers do not extend laterally beyond the wall-contacting/wall-approaching ends of the probe arms. In a subvariation, each of the flexible tether lines connects the wall-contacting/wall-approaching end of an optical probe arm to a radially central position on the catheter having at least substantially the same lateral position as the wall-contacting/wall-approaching end of the probe arm. Thus, in this subvariation, the tether lines are at least substantially perpendicular with the central axis of the catheter.

In any of the embodiments, the catheter may be sized and configured for interrogation of human coronary arteries.

The invention also provides intravascular diagnostic systems that include: an intravascular catheter embodiment of the invention; a power source operably connected to the IVUS imaging element; an ultrasound signal analyzer operably connected to the IVUS imaging element; a light source in optical communication with the optical probe element(s) of the probe arms for illumination of a target region; and a light detecting device in optical communication with the optical probe element(s) of the probe arms for analysis of light collected from the target region. In one variation the light source is a laser, and the light detecting device is a Raman spectrometer, such as a Raman spectrometer configured to measure wavenumber shifted light in the high wavenumber region and/or the fingerprint region. In another variation, the light source is a laser and the light detecting device is a fluorescence spectrometer, such as a time-resolved fluorescence spectrometer.

The invention also provides methods for evaluating blood vessels using the catheters and systems of the invention.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1A shows a basket-style intravascular catheter embodiment of the invention in which the dimension of the probe arms within the field-of-view of the IVUS imaging element is reduced.

FIG. 1B is a side view of a probe arm of the embodiment of FIG. 1A.

FIG. 1C is a top view of a probe arm of the embodiment of FIG. 1A.

FIG. 1D shows a portion of a probe arm in which the height of the probe arm is constant while its width is reduced in the portion of the probe arm that is within the field-of-view of the IVUS imaging element.

FIG. 2 shows an intravascular catheter embodiment of the invention having radially extendable, wall-contacting/wall-approaching optical probe arms, the distal ends of which are tethered to the catheter from a position distally beyond the distal ends of the probe arms.

FIG. 3 shows an intravascular catheter embodiment of the invention having radially extendable, wall-contacting/wall-approaching optical probe arms, the distal ends of which are tethered to the catheter from a position proximal to the distal ends of the probe arms.

FIG. 4 shows an intravascular catheter embodiment of the invention that is similar that of FIG. 2, but in which the IVUS imaging element has a field-of-view diagonally oriented toward the wall-contacting/wall-approaching ends of the probe arms.

FIG. 5 shows a diagnostic catheter system embodiment of the invention.

FIG. 6 shows Raman spectra of cholesterol and various cholesterol esters in the Raman high wavenumber region.

### DETAILED DESCRIPTION

The invention provides intravascular diagnostic catheters including a centrally disposed (e.g., on or around a shaft of the catheter) intravascular ultrasound (IVUS) imaging element and radially extendable, wall-contacting/wall-approaching optical probe arms, where the probe arms are of a novel design for improving the IVUS imaging aspect of the combined IVUS and optical analysis.

One embodiment of the invention provides a basket-type intravascular diagnostic catheter comprising a basket section having radially separated, wall-contacting/wall-approaching, optical probe arms (e.g., 1, 2, 3, 4, 5, 6, 7 or 8 probe arms) and an IVUS imaging element centrally disposed (with respect to radius) within the basket section, in which the thickness of the probe arms is thinned in at least part of the portion of the probe arm which is in the field-of-view of the IVUS imaging element in comparison to the rest of the probe arm. In this manner the shadow cast onto the IVUS image by the probe arm is minimized. The catheter may, for example, be a basket-style catheter including a basket section that includes at least one probe arm, such as at least two probe arms. Thus, although basket style catheters typically have at least two probe arms, basket type catheters having only one radially extendable probe arm are also provided by the invention.

An additional advantage of basket catheter embodiments of the invention, especially those including 3 or more probe arms, is that such configurations tend to self-center the radial viewing IVUS element, thereby permitting and providing for more consistent focusing at depths just beyond the probe arms, i.e., better focusing on and in a blood vessel wall.

Another embodiment provide an intravascular diagnostic catheter in which, rather than typical probe arms of a conventional basket-style catheter, what is provided is one or more radially extendable optical probe arms where the distal end (or near the distal end) of the probe arm is the most radially disposable part of the probe arm and the distal end of the probe arm is connected to the catheter by a tether element, such as a flexible tether element, that is substantially thinner than the probe arm itself. The tether element may or may not limit or be used to control the radial extension of the probe arm. Here again, the catheter is configured so that at least part of the field-of-view of the centrally disposed IVUS element coincides with the thin tether elements, rather than the thicker probe arms, in order to minimize the shadow cast onto the IVUS image by the probe arm.

Still another embodiment of the invention provides an intravascular diagnostic catheter that includes a centrally disposed (with respect to radius) IVUS imaging element and one or more radially extendable optical probe arms where the distal end (or near the distal end) of the probe arm is the most radially disposable part of the probe arm and the distal end of the probe arm is connected to the catheter by a tether element, such as a flexible tether element, in which the probe arms at least substantially do not impinge on the field-of-view of the IVUS imaging element and tethers that are at least substantially not within the field-of-view of the IVUS imaging element are used to stabilize/control the probe arms, thereby providing a clear field-of-view for the IVUS imaging element. A tether may, for example, attach at or near the distal end (optical probe end) of a probe arm and then perpendicularly intersect a central position on the catheter or laterally proceed in the direction of the opposite end of the probe arm before intersecting a central position on the catheter, while the IVUS imaging element is laterally located beyond the distal end (optical probe end) of the probe arm.

Another aspect of the invention for improving the performance of a combined IVUS/Raman diagnostic catheter, which may be employed separately or in combination with other embodiments of the invention, involves using an IVUS imaging element having a field-of-view that is diagonally incident on the target tissue, rather than perpendicularly incident. In one embodiment, the IVUS imaging element of the catheter is angled so that its field-of-view is diagonally incident upon the target tissue and the field-of-view of the Raman spectroscopy probes of the probe arms so that both IVUS imaging data and Raman spectroscopy data can be easily obtained for the same tissue regions. Thus, depending on the type of IVUS imaging element used, the transducer or lateral deflector may be angled so that the ultrasound field of transmission/view is diagonally incident upon the target tissue. This angular configuration generally provides better quality ultrasound imaging and allows the plane in which the Raman spectroscopy measurements are taken and that in which the IVUS signals are collected to be nearer to each other, and even to overlap, thereby providing improved spatial registration of the Raman and IVUS data.

The various aspects of the invention are further described below with respect to the appended drawings.

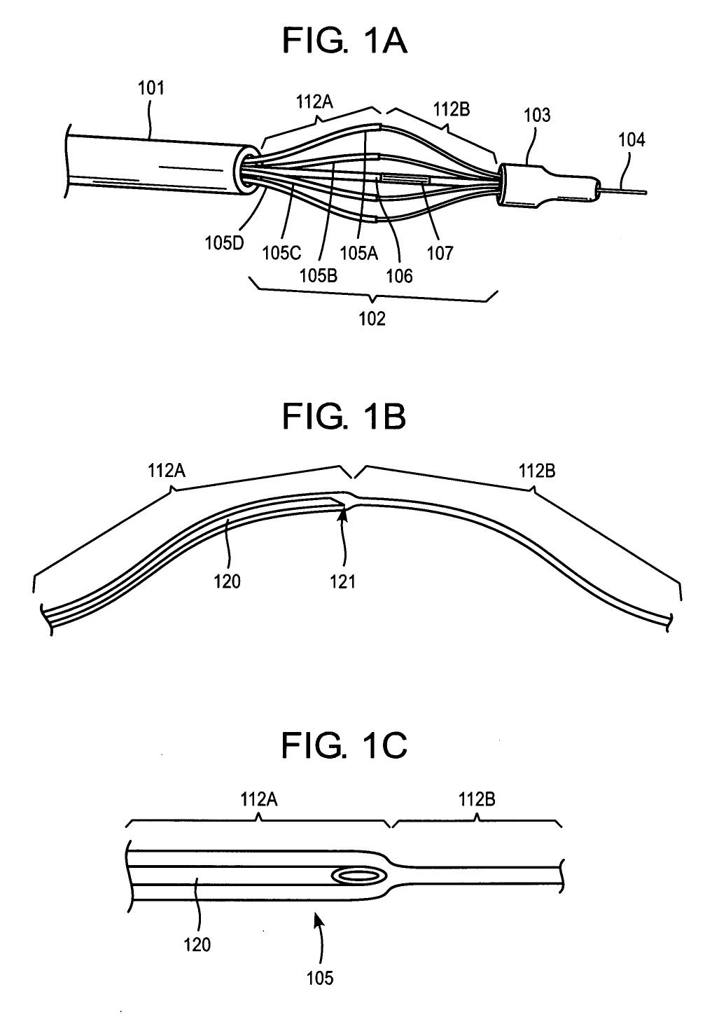

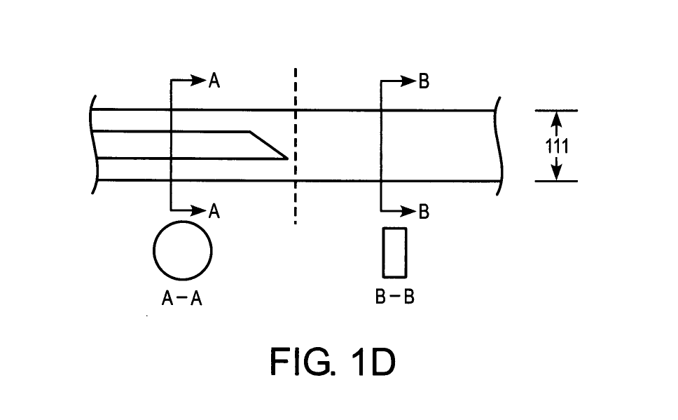

FIG. 1A illustrates a basket-style intravascular catheter embodiment of the invention in which the distal “half” of each probe arm 112B is significantly narrowed in comparison to the proximal half 112A of the probe arm, which contains one or more optical fibers for optical interrogation of a target region. The catheter includes an outer shaft 101, a basket section 102 including a central shaft 106 with a radially viewing IVUS imaging element 107 and four radial extendable optical probe arms 105A-D, and a distal tip 103. Central shaft 106 is hollow to pass a guide wire 104, which is shown extending out of distal tip 103. Rather than using a probe arm of uniform thickness, in the distal half 112B of the probe arm where space for internal optical components is not required, the cross-sectional dimension of the probe arm is minimized. The field-of-view of the IVUS imaging element passes through the thinned distal end of the probe arm. Thus, the shadow or “blind spot” cast by the probe arm on collected IVUS images is reduced, thereby providing better IVUS imaging.

FIG. 1B shows a side view of a probe arm 105 of the catheter embodiment shown in FIG. 1A. What is shown is the junction portion between the thicker proximal section 112A that includes an optical fiber 120 having a distal end 121 configured for side (radial) viewing of a vessel wall and the distal section of the probe arm 112B having a thinner dimension than the proximal section in order to minimize interference with IVUS imaging by the IVUS imaging element. FIG. 1C shows a top view of a probe arm 105 of the catheter embodiment shown in FIG. 1A

FIG. 1D shows a variation probe arm design in which the height dimension 111 of the probe arm is constant while its width is reduced in the portion of the probe arm that is within the field-of-view of the IVUS imaging element. Cross-section A-A of the optical fiber portion of the probe arm is wider, and circular as shown, while cross section B-B is relatively narrower, and rectangular as shown.

Similar embodiments in which the thinner part of the probe arm is located proximally to the thicker, optical element-containing portion of the probe arm are also provided by and within the scope of the invention. In this case, the optical fibers in the distal portion of the probe arm may run distally to the distal tip portion of the catheter to enter the central shaft of the catheter, and thereafter run proximally to the proximal end of the catheter.

Another embodiment of the invention or variation of any of the other embodiments of the invention provides that at least the portion of the probe arm(s) that is within the field-of-view of the IVUS imaging element is composed of material the facilitates acoustic imaging therethrough such as a material having a similar acoustic impedance as living tissue, for example, polymethylpentene (TGX®).

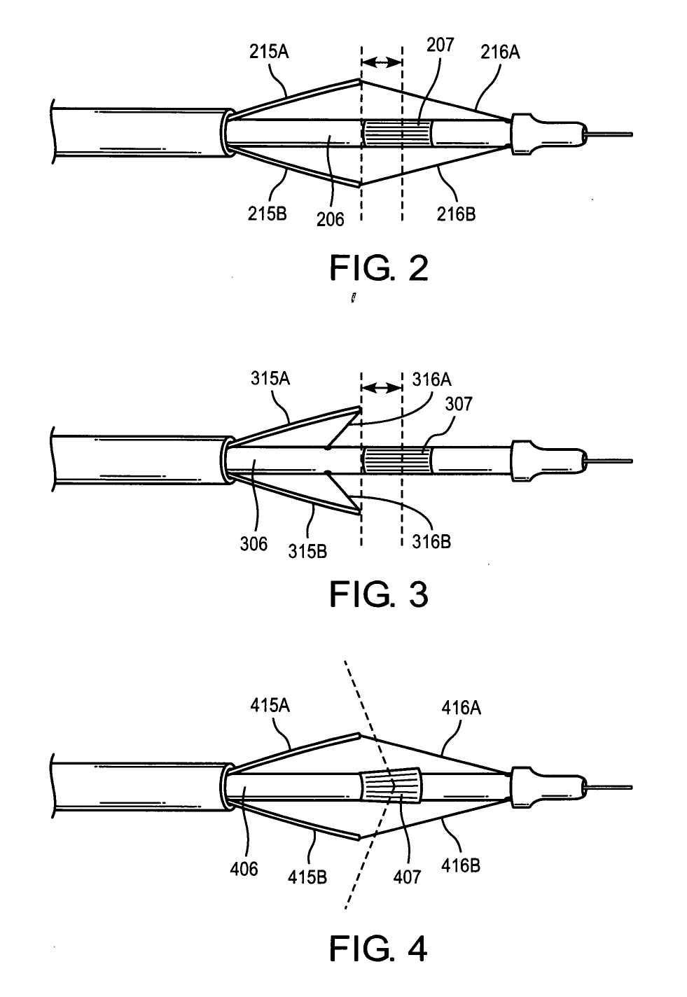

FIG. 2 shows a catheter embodiment of the invention comprising tethered probe arms. Radially extendable, wall-contacting/wall-approaching probe arms 215A and 215B each include one or more optical fibers terminating distally in a side-viewing configuration/assembly for performing optical analytical analyses, such as Raman spectroscopy, of a vessel wall. The IVUS imaging element 207 is disposed on central shaft 206 of the catheter, located slightly distally to the distal end of the probe arms in their radially extended state. A flexible tether line, 216A and 216B, which may, for example, be a high strength flexible polymeric line is connected to the distal end of each probe arm at its proximal end and to the central shaft or distal tip of the catheter at its distal end. The tether lines may optionally enter and run down the inner shaft to the proximal end catheter so that tension on the tether lines may be adjusted. IVUS imaging element 207 is longitudinally disposed so that its field-of-view passes through the very small dimension of the tether lines rather than the thicker dimension of the probe arms. In this manner, obstruction of the field-of-view of the IVUS imaging element is kept to a minimum while the probe arms are provided with positional stabilization and/or control.

FIG. 3 shows a catheter embodiment of the invention that is similar to that shown in FIG. 2, except that in FIG. 3 the tether lines 316A and 316B are attached to or enter the central shaft of the catheter at a longitudinal position proximal to the IVUS imaging element 307 (rather than distal to it as shown in FIG. 2). In this manner, the field-of-view of the IVUS imaging element is left unobstructed.

FIG. 4 shows a catheter embodiment of the invention that is similar to that shown in FIG. 3, except that the IVUS imaging element 407 has a field-of-view that is diagonally oriented in the proximal direction. As shown by the dashed lines, in this manner, the field-of-view of the centrally located IVUS imaging element is brought longitudinally closer the planar field-of-view of the optical probe elements at the distal tips of each probe arm (415A and 415B), thereby improving the registration and integration of the IVUS and Raman spectroscopy data. The diagonally oriented field-of-view of the IVUS element also tends to provide a clearer ultrasound image of a target region in comparison to perpendicularly oriented (head-on) imaging.

As shown in FIGS. 2-4, the IVUS imaging element is located laterally adjacent to the wall-contacting/wall-approaching ends of the probe arms in their radially extended (working) state/configuration, i.e., laterally beyond the said wall-contacting/wall-approaching ends with reference to the lateral direction moving from the catheter ends of the probe arms to the wall-contacting/wall-approaching ends thereof.

Any suitable types of IVUS imaging elements, such as those known in the art, may be used according to the invention. For example, radial scanning, by mechanical rotation, such as of the ultrasound transducer itself or of a lateral beam deflector that laterally deflects ultrasound from/to a stationary transducer may be used. The lateral deflector may, for example, include a 45-degree acoustic mirror face. Rotation may, for example, be provided by a rotating shaft that runs the length of the catheter and/or by a miniature motor (e.g., a MEMS motor) operably connected to the ultrasound transducer or lateral beam deflector. Alternatively, a phase array-type IVUS imaging element may, for example, be used for radial scanning. In addition, any suitable type of ultrasound transducer may be used in implementing the invention. For example, transducers made from conventional piezoelectric materials may be used and newer types of high speed transducer such as capacitive micromachined ultrasonic transducers (CMUTs) and those made from ceramic-based materials may be used.

Generally, the optical probe arm(s) of embodiments of the invention will have disposed therein on or more optical fibers forming the optical probe element thereof. Any suitable sort of side/lateral-viewing optical assembly(ies) may be used to provide a side-viewing optical probe element and numerous sorts of side viewing optics are well known in the art. For example, a 45-deg (or other angle) mirror face or a prism can be used to laterally direct/redirect light from an optical fiber. Similarly, an optical fiber can be provided with an angularly faceted tip to direct and receive light that is off-axis with respect to the fiber. The probe arms of basket-type catheters of the invention may generally use side-viewing optical fibers/assemblies. In the non-basket-type catheter embodiments of the invention, front-viewing or side-viewing optical fibers/assemblies may be appropriate depending on the working angle at which the probe arms approach a tissue sample. Catheter embodiments of the invention may include a plurality of radially separate probe arms such as those having 2, 3, 4, 5, 6, 7, 8 or more radially separated probe arms, in order to obtain radially segregated optical measurements that can be integrated with the radial IVUS scan readings.

Optical analytical techniques that may be employed in conjunction with IVUS according to the invention include, for example, Raman spectroscopy such as high wavenumber Raman spectroscopy and/or fingerprint region Raman spectroscopy, laser-induced fluorescence spectroscopy (LIFS), such as time-resolved laser-induced fluorescence spectroscopy (TR-LIFS), absorbance spectroscopy, such as infrared (IR) or near infra-red (NIR) absorbance spectroscopy, interferometry such as optical coherence tomography (OCT) and low-coherence interferometry (LCI), and laser speckle spectroscopy. U.S. Publication No. 20060139633 discloses methods and systems of high-wavenumber Raman spectroscopy for measuring tissue properties including for characterizing atherosclerotic plaques, and is incorporated by reference herein in its entirety. U.S. Pat. No. 6,272,376 discloses methods and systems of time-resolved laser-induced fluorescence spectroscopy, including for identifying and characterizing lipid-rich vascular lesions, and is incorporated by reference herein in its entirety. International Publication No. WO2005019800 discloses methods for fluorescence lifetime imaging microscopy and spectroscopy, including ultra-fast methods for analysis of fluorescence lifetime imaging is also described, facilitating real-time analysis of compositional and functional changes in samples, and is incorporated by reference herein in its entirety. Low-coherence interferometry methods, such as OCT, are disclosed in U.S. Pat. Nos. 7,190,464, 6,903,854 and 6,134,003 and U.S. Publication No. 2005/0020925, each of which is incorporated by reference herein in its entirety. U.S. Pat. No. 7,061,606 and U.S. Pub. No. 20040077950 disclose near-infrared (NIR) spectroscopy, such as analysis of NIR absorbance, transmittance and reflectance spectra, and are incorporated by reference herein in their entireties. U.S. Pub. No. 20020183601 discloses laser speckle-based methods and systems for analyzing tissue, and is incorporated by reference herein in its entirety.

Particularly advantageous is the combination of a chemical composition-determining optical technique such as Raman spectroscopy and/or LIFS, especially TR-LIFS, with physical property determination by IVUS in the present invention.

The invention also provides combined Optical Analysis/IVUS systems that generally include, in addition to a catheter according to the invention, a light source for performing the optical analytical technique and a light detection/analysis unit for analyzing light collected via the catheter as well as a power source for the ultrasound transducer (or pulsed light source in the case of optoacoustic stimulation) and wires/means for collecting and analyzing ultrasound signals from the target tissue. For example, for Raman spectroscopy, the system will include a light source such as a laser, for example, a feedback-stabilized multi-mode laser diode or a single-mode laser and a Raman spectrometer for measuring/analyzing light collected from a target tissue. For LIFS, the system will include a light source such as a laser and a fluorescence spectrometer. For TR-LIFS a spectrometer having temporal resolution may be used. For intereferometry, such as OCT, the system may include a broadband light source such as a superluminescent light-emitting diode or a pico-second pulse laser and an interferometer, such as a Michelson interferometer for analyzing light. One or more computers, or computer processors generally working in conjunction with computer accessible memory, may be part of the system for controlling the various elements and operations of the system and/or for analyzing information obtained by the system.

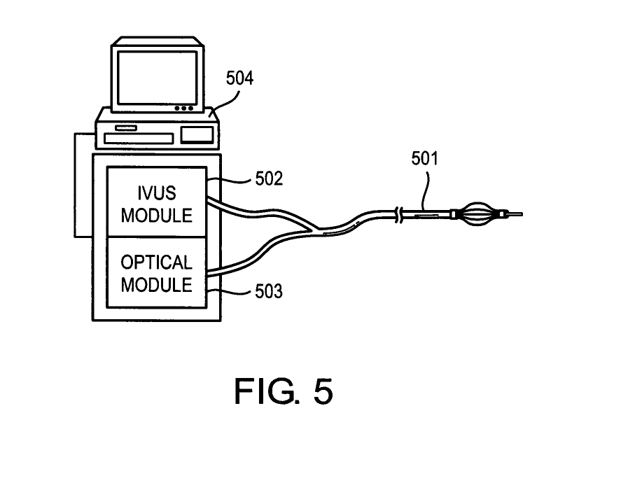

FIG. 5 schematically illustrates a diagnostic catheter system embodiment that includes a catheter 501 that includes one or more wall-contacting/wall-approaching optical probe arms and a centrally disposed radially scanning IVUS element, such as the embodiments shown in FIGS. 1-4, an IVUS module 502 including a power source for the ultrasound transducer and an ultrasound signal analyzer, an optical module 503 including a light source and a light measurement/analysis unit for analyzing collected light, and a computer 504 for controlling the components of the system and analyzing/presenting data obtained via the system. The system embodiments of the invention may also include a catheter pullback drive mechanism (not shown), such as those known in the art, so that IVUS and optical measurements may be obtained during a pullback procedure, in a blood vessel, such as an artery, for example, the coronary artery.

Raman spectroscopy has proven capable of determining the chemical composition of tissues and diagnosing human atherosclerotic plaques. Typical methods of collecting Raman scattered light from the surfaces of artery do not register information about how far the scattering element is from the collection optics. Two wavenumber regions that yield useful information for evaluating the condition of blood vessels are the so-called Raman fingerprint region i.e., approximately 200 to 2,000 cm⁻¹, and the so-called high wavenumber region, i.e., approximately 2,600 to 3,200 cm⁻¹. The collection of Raman spectra in the fingerprint (FP) region, through optical fibers is complicated by Raman “background” signal from the fibers themselves. In order to collect uncorrupted FP spectra, complex optics and filters on the tips of catheters have been required and often these designs necessitated the use of multiple fibers. Since the Raman scattered signal is weak, large multimode fibers are utilized in the multi-fiber catheter designs, which creates an unwieldy catheter that is less than optimal for exploring delicate arteries, such as the human coronary arteries. However, common optical fiber materials generate very little Raman background signal in the high wavenumber region, permitting a simplified, single optical fiber probe element implementation of intravascular Raman spectroscopy.

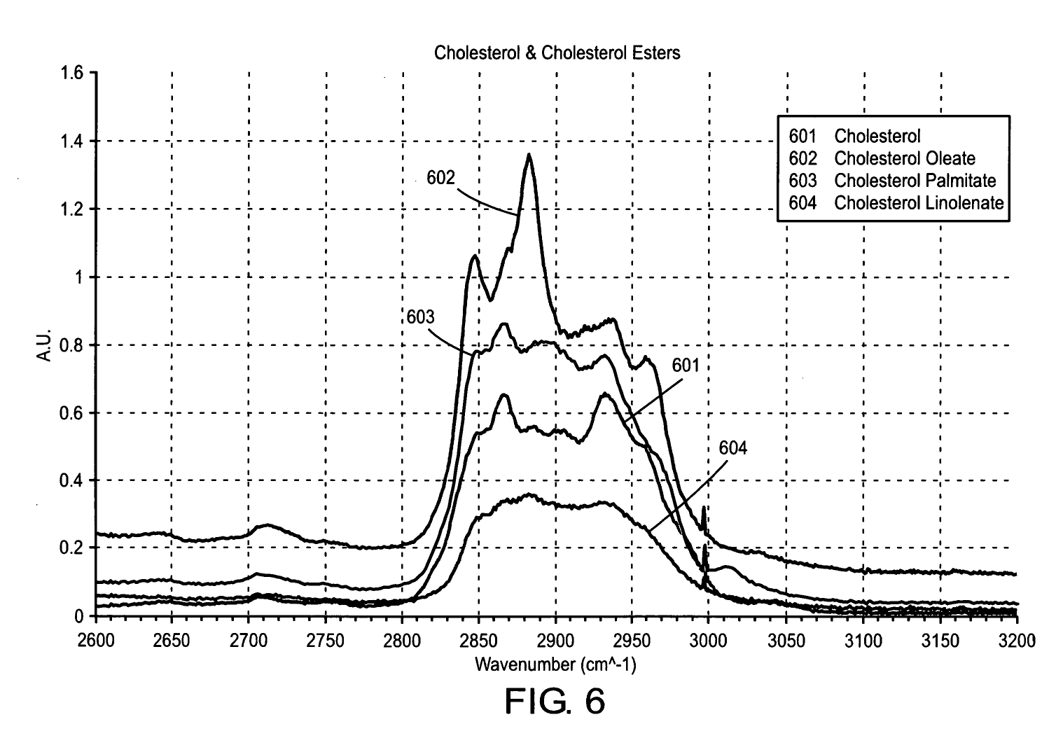

Since cholesterol and its esters have distinctive Raman scattering profiles within the Raman high wavenumber region, the use of the Raman high wavenumber region for analysis is particularly useful for locating and characterizing lipid-rich deposits or lesions as may occur in blood vessels, such a vulnerable plaques in arteries, such as the coronary arteries. FIG. 6 shows Raman spectra of cholesterol and cholesterol esters in the high wavenumber region. Specifically, curve 601 is a Raman spectrum for cholesterol, curve 602 is a Raman spectrum for cholesterol oleate, curve 603 is a Raman spectrum for cholesterol palmitate and curve 604 is a Raman spectrum for cholesterol linolenate.

A related embodiment of the invention provides a catheter system for the evaluation of blood vessel walls that includes an intravascular diagnostic catheter as described herein, a light source such as a laser for stimulating Raman scattered light emissions from a target region via the wall-contacting/wall-approaching portion/end of the probe arms of the catheter, a Raman spectrometer for analyzing Raman scattered light collected from a target via the wall-contacting/wall-approaching portion/end of the probe arms of the catheter, a power source for driving the ultrasound transducer of the IVUS imaging element of the catheter and an ultrasound analyzer unit for receiving and analyzing the ultrasound signals from a target region. The system may be configured to collect and analyze Raman spectral data within the region of approximately 2,600 to 3,200 cm⁻¹, i.e., the so-called high wavenumber region, and/or the within the region of approximately 200 to 2,000 cm⁻¹, i.e., the so-called fingerprint region. The optical probe arms may, for example, each have a single optical fiber and the system may be configured to perform high wavenumber Raman spectroscopy from each probe arm via the single optical fiber.

Advantageously, the system may be configured to provide depth-resolved chemical composition information about a target based on Raman spectroscopic data and intravascular ultrasound data obtained from interrogating the target using the intravascular diagnostic catheter. One embodiment of the invention utilizes Raman scattered light shifted in the high wavenumber (HW) region, i.e., approximately 2,600 to 3,200 cm⁻¹, and combines this information with IVUS data collected via the IVUS imaging element to provide chemical compositional information as a function of depth in a lumen wall, such as a blood vessel wall, such as an artery wall.

The invention also generally provides methods for evaluating the condition of a blood vessel such as an artery, such as a human coronary artery, using an intravascular catheter and/or intravascular catheter system according to the invention to interrogate the wall of the vessel using IVUS alone or IVUS in combination with an optical analytical technique such as Raman spectroscopy or fluorescence spectroscopy. Atherosclerotic lesions and lipid rich deposits and/or lesions, such as vulnerable plaques, may be located and/or characterized in a blood vessel such as an artery by interrogating the blood vessel wall by IVUS alone or IVUS in combination with an optical analytical technique such as Raman spectroscopy or fluorescence spectroscopy using a catheter or catheter system according to the invention.

One embodiment of the invention provides a method for evaluating the wall of a blood vessel such as an artery, such as a coronary artery, such as a human coronary artery, that includes the steps of:
providing any of the intravascular catheter embodiments of the invention having a proximal end and a distal insertion end including at least one, such as at least two, radially extendable wall-contacting/wall-approaching optical probe arm and a centrally disposed IVUS imaging element;
disposing the distal end of the catheter in a blood vessel; and
taking both ultrasound readings and optical readings of the vessel wall at one or more locations in the blood vessel using the IVUS imaging element and the at least one optical probe arm.

A related embodiment of the invention provides a method for evaluating the wall of a blood vessel such an artery, such as a coronary artery, such as a human coronary artery, that includes the steps of:
providing any of the intravascular basket catheter embodiments of the invention having a proximal end and a distal insertion end including a basket section comprising at least one, such as at least two, radially extendable wall-contacting/wall-approaching optical probe arms and a centrally disposed IVUS imaging element within or adjacent to the basket section;
disposing the basket section of the catheter in a blood vessel; and
taking both ultrasound readings and optical readings of the vessel wall at one or more locations in the blood vessel using the IVUS imaging element and the at least one optical probe arms.

It should be understood for the above methods that the probe arms are radially extended to contact or closely near the vessel walls in order to take readings from the probe arm-disposed optical assemblies. Thus, the probe arms and, in particular, the portion including the optical probe element(s) may be configured to contact or approach the vessel wall. As used herein, the term “wall-approaching” means that the probe arm and the probe element portion thereof in particular is configured to near the vessel wall and/or contact the vessel wall. It will be readily recognized by those knowledgeable in the art that one or more probe arms may be in contact with a vessel wall at one time and not at another during the course of a procedure due to the changing geometry of a subject blood vessel and the present invention is intended to cover all such situations. The step of taking readings may include taking the recited readings at more than one longitudinal location in a blood vessel, for example, while the catheter is pulled back by operation of a catheter pullback mechanism.

Each of the patents and other publications cited in this disclosure is incorporated by reference in its entirety.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. An intravascular interrogation catheter, comprising:
a proximal end and a distal insertion end;
a basket section disposed at or near the distal insertion end of the catheter, said basket section comprising at least one radially extendable wall-approaching optical probe arms each having a proximal end and a distal end and a wall-approaching portion disposed therebetween and each having at least one optical fiber entering an end of the probe arm and terminating in the wall-approaching portion thereof, the optical fibers of each probe arm all entering from the distal ends of the probe arms or all entering from the proximal ends of the probe arms;
a radially centrally disposed, radially scanning IVUS imaging element disposed within the basket section and having a field-of-view,
wherein the probe arms extend laterally over the IVUS imaging element, a first lateral portion of each probe arm being in the field-of-view of the IVUS imaging element and a second lateral portion of each probe arm being outside of the field-of-view of the IVUS imaging element, and
wherein the cross-sectional dimension of the first lateral portion of the at least one probe arm is smaller than the cross-sectional dimension of the second lateral portion of the probe arms.

2. The catheter of claim 1, wherein the catheter is sized and configured for interrogation of human coronary arteries or human carotid arteries.

3. An intravascular interrogation catheter, comprising:
a proximal end and a distal insertion end;
an interrogation section disposed at or near the distal insertion end of the catheter, said interrogation section comprising at least one radially extendable wall-approaching optical probe arms each having a wall-approaching portion disposed at an end thereof and each having at least one optical fiber entering the probe arm and terminating at or near the wall-approaching end,
a radially centrally disposed, radially scanning IVUS imaging element laterally adjacent to the wall-approaching ends of the optical probe arms in their radially extended state,
wherein the wall-approaching end of each probe arm is connected by a flexible tether line to a radially central position on the catheter.

4. The catheter of claim 3, wherein each of the flexible tether lines laterally traverses the IVUS imaging element before connecting to a radially central position of the catheter and wherein the cross-sectional dimension of the flexible tether lines is smaller than the cross-sectional dimension of the optical probe arms.

5. The catheter of claim 3, wherein each of the flexible tether lines connects the wall-approaching end of an optical probe arm to a radially central position on the catheter having a lateral position laterally within the lateral dimension of the probe arm in its radially extended state.

6. The catheter of claim 3, wherein each of the flexible tether lines connects the wall-approaching end of an optical probe arm to a radially central position on the catheter having at least substantially the same lateral position as the wall-approaching end of the probe arm.

7. The catheter of claim 3, wherein the tether lines are extendable and retractable to control the probe arms.

8. The catheter of claim 4, wherein the tether lines are extendable and retractable to control the probe arms.

9. The catheter of claim 5, wherein the tether lines are extendable and retractable to control the probe arms.

10. The catheter of claim 6, wherein the tether lines are extendable and retractable to control the probe arms.

11. The catheter of claim 3, wherein the catheter is sized and configured for interrogation of human coronary arteries.

12. An intravascular interrogation system, comprising:
an intravascular catheter according to claim 1;
a power source operably connected to the IVUS imaging element;
an ultrasound signal analyzer operably connected to the IVUS imaging element;
a light source in optical communication with the optical probe element for illumination of a target region; and
a light detecting device in optical communication with the optical probe element for analysis of light collected from the target region.

13. The system of claim 12, wherein
the light source is a laser, and
the light detecting device is a Raman spectrometer.

14. The system of claim 13, wherein the Raman spectrometer is configured to measure wavenumber-shifted light in the high wavenumber region.

15. The system of claim 13, wherein the Raman spectrometer is configured to measure wavenumber-shifted light in the fingerprint region.

16. The system of claim 12, wherein
the light source is a laser, and
the light detecting device is a fluorescence spectrometer.

17. An intravascular interrogation system, comprising:
an intravascular catheter according to claim 3;
a power source operably connected to the IVUS imaging element;
an ultrasound signal analyzer operably connected to the IVUS imaging element;
a light source in optical communication with the optical probe element for illumination of a target region; and
a light detecting device in optical communication with the optical probe element for analysis of light collected from the target region.

18. The system of claim 17, wherein
the light source is a laser, and
the light detecting device is a Raman spectrometer.

19. The system of claim 18, wherein the Raman spectrometer is configured to measure wavenumber-shifted light in the high wavenumber region.

20. The system of claim 18, wherein the Raman spectrometer is configured to measure wavenumber-shifted light in the fingerprint region.

21. The system of claim 17, wherein
the light source is a laser, and
the light detecting device is a fluorescence spectrometer.
